(19) United States
(12) Patent Application Publication
(10) Pub. No.: US 2025/0000402 A1
(43) Pub. Date: Jan. 2, 2025

(54) Dual Chamber Specimen Collection Container Assembly

(71) Applicant: Becton, Dickinson and Company, Franklin Lakes, NJ (US)

(72) Inventors: Arun U. Nair, Denville, NJ (US); Shruti Wigh, Maywood, NJ (US)

(21) Appl. No.: 18/693,257

(22) PCT Filed: Sep. 16, 2022
(86) PCT No.: PCT/US2022/043750

§ 371 Date: Mar. 19, 2024

(51) Int. Cl. A61B 5/15; B01L 3/00

(52) U.S. Cl. CPC A61B 5/150022; A61B 5/150251; A61B 5/150343; A61B 5/150755; B01L 3/5021; B01L 2200/025; B01L 2300/046; B01L 2300/0838; B01L 2300/0858

(57) ABSTRACT

A specimen collection container assembly including a collection tube and an interior reservoir formed within the collection tube. The interior reservoir includes an upper chamber, a lower chamber, and an elongated channel fluidly coupling the upper chamber and the lower chamber. A diameter of the elongated channel is smaller than both a diameter of the upper chamber and a diameter of the lower chamber. The assembly further includes a bead-type separator which is configured to plug an upper neck portion between the upper chamber and the elongated channel. The bead-type separator is insertable into the interior reservoir after a specimen sample is collected and the collection tube is subjected to centrifugation.

### CROSS-REFERENCE TO RELATED APPLICATION

The present application claims priority to U.S. Provisional Application Ser. No. 63/246,496, entitled “Dual Chamber Specimen Collection Container Assembly”, filed Sep. 21, 2021, the entire disclosure of which is hereby incorporated by reference in its' entirety.

### BACKGROUND OF THE INVENTION

### Field of the Invention

The present disclosure relates to a small volume specimen collection container assembly for the collection, storage, and transfer of a blood or specimen sample obtained from a patient for medical diagnostic testing. More specifically, the present disclosure relates to a dual chamber collection container for the collection of blood samples, wherein the collection container may incorporate a bead-type separator to maintain reliable barrier separation of the blood components post-centrifugation and/or prevent unwanted insertion of a probe or other device beyond a desired chamber within the collection container.

### Description of Related Art

Conventional specimen collection devices according to the prior art (e.g., capillary blood collection devices) typically provide a microtube or collection container having a receiving lip or funnel feature that engages the skin surface of a patient that has been pierced so as to draw a blood sample from the capillaries located just beneath the skin surface. The internal collection cavity or reservoir of such prior art collection containers is typically much smaller than the overall volume of the specimen collection container, as the volume of blood or specimen collected is relatively low (e.g., 800 μL or less). However, the larger overall volume of the collection container allows for compatibility with certain automated processes employed both before and after a specimen is collected, such as, e.g., sorting, centrifugation, analysis, sealing, etc.

As is known in the art, upon centrifugation of a specimen collection container holding a blood sample, the primary components of the blood (i.e., the plasma/serum and the hematocrit comprised primarily of red blood cells) separate by density, with the denser hematocrit settling at the bottom of the interior reservoir, and the less dense plasma/serum collecting thereabove. In many instances, a gel separator substance is also provided in the collection reservoir. The gel separator substance is configured to have a density between that of the plasma/serum and hematocrit. Accordingly, upon centrifugation, the gel separator substance forms a barrier between the plasma/serum and the hematocrit.

However, while the gel separator substance may typically form an effective barrier between the separated blood components, the physical properties of the gel separator limit its effectiveness as a physical barrier. Thus, when a probe is inserted into the container for sampling and analysis of the plasma/serum, the probe may inadvertently penetrate the hematocrit layer, potentially resulting in a contaminated or inaccurate sample. Additionally, the gel separator may not provide a suitable barrier to maintain separation of the blood components during transport and/or storage.

### SUMMARY OF THE INVENTION

Accordingly, a need exists for a specimen collection container assembly having a dual chamber configuration to physically isolate the separate blood components. Additionally, there exists a need for a bead-type or similar separator component to maintain separation of the blood components during transport and/or storage, as well as to prevent unwanted penetration of a probe or other device into the hematocrit layer of the separated blood sample.

In accordance with an embodiment of the present disclosure, a specimen collection container assembly includes a collection tube, an interior reservoir formed within the collection tube, wherein the interior reservoir includes an upper chamber, a lower chamber, and an elongated channel fluidly coupling the upper chamber and the lower chamber. A diameter of the elongated channel is smaller than both a diameter of the upper chamber and a diameter of the lower chamber. The interior reservoir further includes a bead-type separator, wherein the bead-type separator is configured to plug an upper neck portion between the upper chamber and the elongated channel, and further wherein the bead-type separator is insertable into the interior reservoir after a specimen sample is collected and the collection tube is subjected to centrifugation.

In certain configurations, the bead-type separator is ellipsoidal in shape. The bead-type separator may be formed of a hard material. The bead-type separator may be sized and configured to plug the upper neck portion by way of fluid pressure from a specimen sample collected within the interior reservoir. Optionally, the bead-type separator may be sized and configured to plug the upper neck portion by way of a friction fit with an interior surface of the elongated channel proximate the upper neck portion.

In certain configurations, an overall volume of the lower chamber is configured according to a total volume of a hematocrit portion of a specimen sample collectable within the interior reservoir. Optionally, the collection tube further includes an open lip portion. The open lip portion is at least partially formed as a scoop to accommodate the collection of a capillary blood sample into the interior reservoir.

In accordance with an embodiment of the present disclosure, a specimen collection container includes a collection tube, an interior reservoir formed within the collection tube, wherein the interior reservoir includes an upper chamber, a lower chamber, and an elongated channel fluidly coupling the upper chamber and the lower chamber, wherein a diameter of the elongated channel is smaller than both a diameter of the upper chamber and a diameter of the lower chamber. An overall volume of the lower chamber is configured according to a total volume of a hematocrit portion of a maximum specimen sample volume collectable within the interior reservoir.

In certain configurations, the elongated channel includes an upper neck portion providing the transition between the upper chamber and the elongated channel. The elongated channel may include a lower neck portion providing a transition between the lower chamber and the elongated channel. The collection tube may include an open lip portion. The open lip portion may be at least partially formed as a scoop to accommodate the collection of a capillary blood sample into the interior reservoir.

In accordance with yet another embodiment of the present disclosure, a method of collecting and analyzing a capillary blood sample includes providing a specimen collection container including a collection tube, an interior reservoir formed within the collection tube, wherein the interior reservoir includes an upper chamber, a lower chamber, and an elongated channel fluidly coupling the upper chamber and the lower chamber. A diameter of the elongated channel is smaller than both a diameter of the upper chamber and a diameter of the lower chamber. The method also includes collecting the capillary blood sample within the specimen collection container such that the lower chamber, the elongated channel, and at least a portion of the upper chamber are filled with the capillary blood sample. The method further includes subjecting the specimen collection container to centrifugation, and inserting a bead-type separator into the specimen collection container after centrifugation in order to plug an upper neck portion of the elongated channel between the upper chamber and the elongated channel.

In certain configurations, the method further includes inserting a probe into the specimen collection container to collect a sample from a plasma/serum portion of the capillary blood sample. The probe may be restricted from being inserted beyond the upper chamber of the specimen collection container by the bead-type separator.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is side cross-sectional view of a dual chamber specimen collection container in accordance with an aspect of the present disclosure;

FIG. 2A is a side cross-sectional view of a specimen collection container assembly in accordance with an aspect of the present disclosure in a pre-centrifugation condition;

FIG. 2B is a side cross-sectional view of the specimen collection container assembly of FIG. 2A in a first post-centrifugation condition;

FIG. 2C is a side cross-sectional view of the specimen collection container assembly of FIG. 2A in a second post-centrifugation condition; and

FIG. 3 is a flowchart of a method of blood sample collection and analysis using a specimen collection container assembly in accordance with an aspect of the present disclosure.

### DETAILED DESCRIPTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

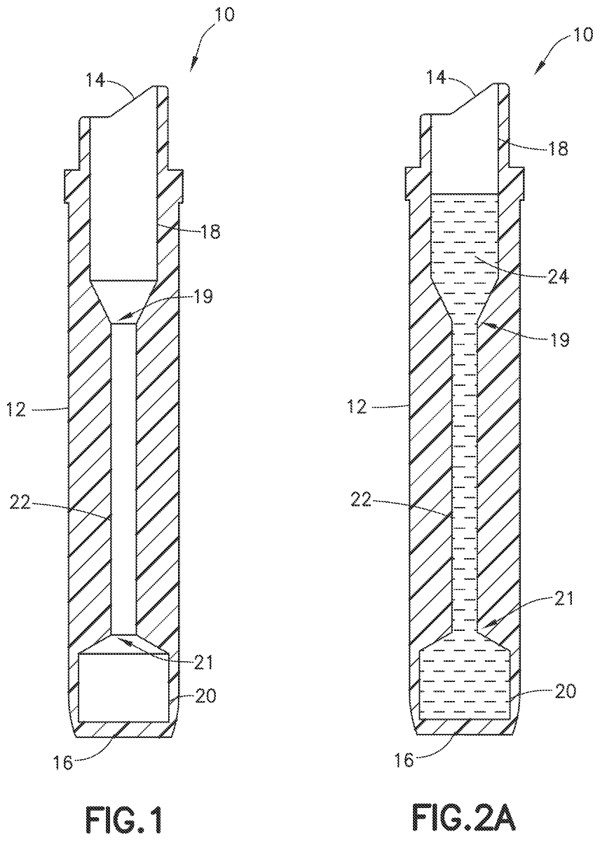

Referring to FIG. 1, a specimen collection container assembly 10 in accordance with one aspect of the present disclosure is shown. As shown in FIG. 1, specimen collection container assembly 10 comprises a collection tube defined by an exterior sidewall 12, an open lip portion 14, and a closed bottom portion 16. In some embodiments, the open lip portion 14 may be partially raised or otherwise formed into a “scoop” so as to aid the collection of capillary blood. However, it is to be understood that open lip portion 14 is not limited to such a configuration.

In some embodiments, the specimen collection container assembly 10 is configured as a microtube suited for capillary collection of blood samples having overall exterior dimensions conforming to a standard 13 mm×75 mm tube so as to be compatible with standard testing instruments and/or automation processes. The collection tube may be formed by, e.g., injection molding, from suitable plastic or composite material as is known to be suitable by those of ordinary skill in the art.

Referring still to FIG. 1, specimen collection container assembly 10 includes an interior reservoir formed of two separate primary chambers: an upper chamber 18 and a lower chamber 20. An elongated channel 22 fluidly connects the upper chamber 18 and the lower chamber 20, with an upper neck portion 19 providing the transition between the upper chamber 18 and the elongated channel 22, and a lower neck portion 21 providing the transition between the lower chamber 20 and the elongated channel 22. As will be described in further detail below, the overall size, shape, and separation of the upper chamber 18, the lower chamber 20, and the elongated channel 22 is such that a typical capillary blood specimen, even while relatively low in volume (e.g., 800 μL or less), is sufficient to fill the lower chamber 20, fill the elongated channel 22, and at least partially fill the upper chamber 18. In some embodiments, an overall volume of the lower chamber 20 is configured according to a total volume of a hematocrit portion of a maximum specimen sample volume collected within the interior reservoir of the specimen collection container assembly 10. In this way, the lower chamber 20 may be configured to hold only the hematocrit portion of a blood specimen sample, while the upper chamber 18 and at least a portion of elongated channel 22 may be configured to hold a plasma/serum portion of the blood specimen sample.

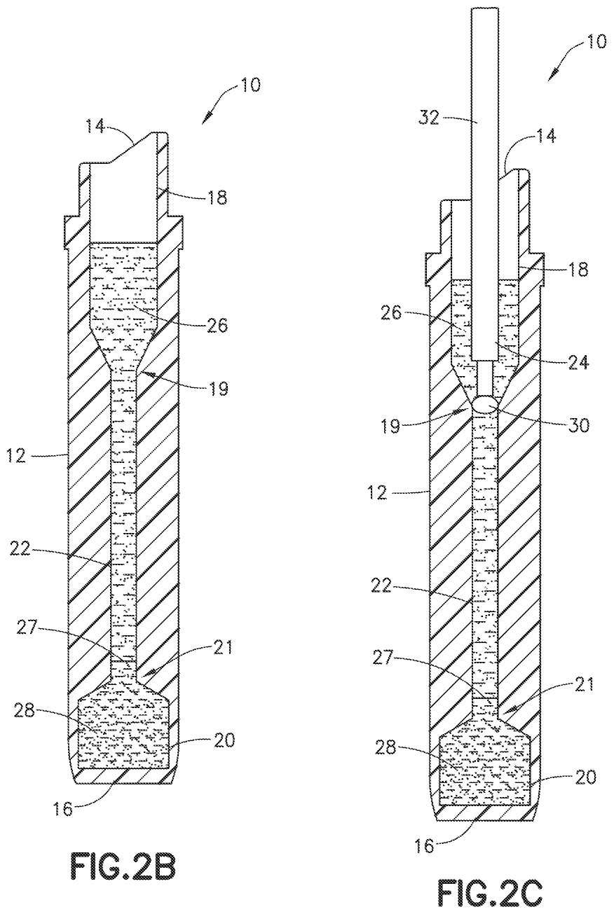

Referring now to FIGS. 2A-2C, specimen collection container assembly 10 is shown in various pre- and post-centrifugation states in accordance an aspect of the present disclosure. First, FIG. 2A illustrates specimen collection container assembly 10 in a pre-centrifugation state, with a whole blood sample 24 collected therein. As described above, the whole blood sample 24 is large enough in volume to fill the lower chamber 20 and the elongated channel 22, and at least partially filling the upper chamber 18. The whole blood sample 24 may be, e.g., a capillary blood sample collected from a patient at the site of a lancet puncture.

Next, FIG. 2B illustrates specimen collection container assembly 10 in a first post-centrifugation state. That is, upon centrifugation, the whole blood sample 24 shown in FIG. 2A separates into two primary component parts: a plasma/serum portion 26 and the hematocrit portion 28, with a transition point 27 delineating the respective component parts. The denser hematocrit portion 28 is forced to the bottom of the interior reservoir such that the hematocrit portion 28 settles primarily in the lower chamber 20. In some embodiments, the size and shape of the lower chamber 20 is specifically configured such that the hematocrit portion 28 of a typical blood sample fills the lower chamber 20, while avoiding (or minimizing) entrance of the hematocrit portion 28 into the elongated channel 22. While not shown in FIG. 2B, it is to be understood that a cap or other closure is provided on specimen collection container assembly 10 after collection of the sample and during the centrifugation process.

As is also shown in FIG. 2B, the plasma/serum portion 26 fills a substantial portion of the elongated channel 22 and the upper chamber 18. In this way, the plasma/serum portion 26 within upper chamber 18 is easily accessible to a probe or other diagnostic tool used for sample analysis. While plasma/serum portion 26 is shown as substantially filling all or most of the elongated channel 22 above the lower neck portion 21, it is to be understood that the present disclosure is not limited as such, and the transition point 27 between the respective component parts of the blood sample may be higher or lower within the elongated channel 22, or even within the lower chamber 20.

Furthermore, while also not shown in FIGS. 2A-2C, in some embodiments, a gel separator substance (e.g., a polyester gel) may also be provided within the specimen collection container assembly 10. As is known in the art, such a gel separator substance would be configured to have a density between that of the plasma/serum portion 26 and the hematocrit portion 28. Thus, upon centrifugation, the gel separator substance would settle at or near the transition point 27 between the component parts of the blood sample, thereby creating an effective barrier between the plasma/serum portion 26 and the hematocrit portion 28.

Next, referring to FIG. 2C, specimen collection container assembly 10 in a second post-centrifugation state is shown. After centrifugation, a substantially solid bead-type separator 30 may be introduced through the open lip portion 14 and into the upper chamber 18. The shape and diameter of the bead-type separator 30 is such that the bead-type separator 30 substantially plugs the upper neck portion 19 between the upper chamber 18 and the elongated channel 22. In this way, a probe 32 introduced into the specimen collection container assembly 10 is prevented from entering the elongated channel 22 and/or the lower chamber 20, thus protecting against the probe 32 inadvertently accessing the hematocrit portion 28 during sample collection, which could contaminate the sample. In one embodiment, the bead-type separator 30 may be maintained in the upper neck portion 19 by way of fluid force from the plasma/serum portion 26 within the upper chamber 18. However, in another embodiment, the bead-type separator 30 may be maintained in the upper neck portion 19 by a friction fit with the interior walls of the elongated channel 22 at the upper neck portion 19.

Additionally, when positioned at the upper neck portion 19, the bead-type separator 30 may act to ensure continued separation between the plasma/serum portion 26 and the hematocrit portion 28 during transport and storage of the specimen collection container assembly 10.

The bead-type separator 30 may be formed of any appropriate material such as, e.g., plastic, polymer, metal, etc. Furthermore, while shown and described with respect to FIG. 2C as being ellipsoidal, it is to be understood that separator 30 may be any appropriate shape capable of blocking the elongated channel 22, such as, e.g., spherical, buoy-shaped, etc. Also, in some embodiments, the separator 30 may be formed of a material that is not a hard solid but is sufficient in blocking the elongated channel 22. For example, in some embodiments, separator 30 may be formed of, e.g., a rubber or gel material.

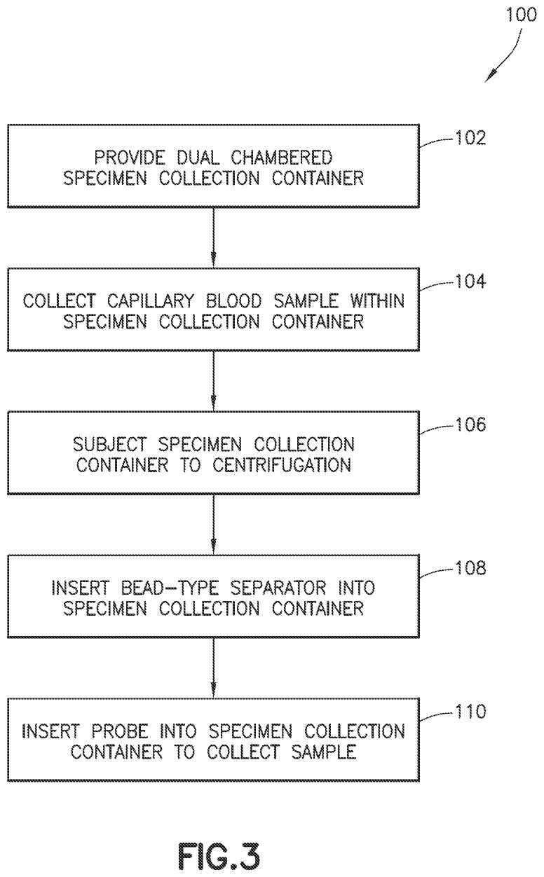

Next, referring to FIG. 3, a method 100 of collecting and analyzing a capillary blood sample in accordance with an aspect of the present disclosure is illustrated. First, at step 102, a dual chambered specimen collection container (such as that described above with respect to FIG. 1) is provided. Then, at step 104, a capillary blood sample is collected within the specimen collection container such that both chambers of the specimen collection container (and the elongated channel fluidly coupling the chambers) are at least partially filled with the blood sample.

At step 106, the specimen collection container is subjected to mechanical centrifugation which, as described above, separates the collected blood sample into its component parts (i.e., a plasma/serum portion and a hematocrit portion). Due to the dual chambered configuration of the specimen collection container, the plasma/serum portion largely collects in an upper chamber of the specimen collection container, while the denser hematocrit portion largely collects in a lower chamber.

Next, at step 108, a bead-type separator is inserted into the specimen collection container. As described above with respect to FIG. 2C, the bead-type separator is sized and configured to block passage between the upper and lower chambers of the specimen collection chamber, thereby providing a physical barrier between the upper and lower chambers. Finally, at step 110, a probe (or other diagnostic device) is inserted into the specimen collection container in order to collect an appropriate sample. Due to the presence of the bead-type separator, collection of the sample is limited to the plasma/serum portion within the upper chamber, thereby avoiding contamination of the sample by inadvertent access by the probe into the hematocrit portion.

While several embodiments of a dual chambered specimen collection container assembly incorporating a bead-type separator were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A specimen collection container assembly comprising:
a collection tube;
an interior reservoir formed within the collection tube, wherein the interior reservoir comprises:
an upper chamber,
a lower chamber, and
an elongated channel fluidly coupling the upper chamber and the lower chamber, wherein a diameter of the elongated channel is smaller than both a diameter of the upper chamber and a diameter of the lower chamber; and
a bead-type separator,
wherein the bead-type separator is configured to plug an upper neck portion between the upper chamber and the elongated channel, and further wherein the bead-type separator is insertable into the interior reservoir after a specimen sample is collected and the collection tube is subjected to centrifugation.

2. The assembly of claim 1, wherein the bead-type separator is ellipsoidal in shape.

3. The assembly of claim 1, wherein the bead-type separator is formed of a hard material.

4. The assembly of claim 1, wherein the bead-type separator is sized and configured to plug the upper neck portion by way of fluid pressure from a specimen sample collected within the interior reservoir.

5. The assembly of claim 1, wherein the bead-type separator is sized and configured to plug the upper neck portion by way of a friction fit with an interior surface of the elongated channel proximate the upper neck portion.

6. The assembly of claim 1, wherein an overall volume of the lower chamber is configured according to a total volume of a hematocrit portion of a specimen sample collectable within the interior reservoir.

7. The assembly of claim 1, wherein the collection tube further comprises an open lip portion.

8. The assembly of claim 7, wherein the open lip portion is at least partially formed as a scoop to accommodate the collection of a capillary blood sample into the interior reservoir.

9. A specimen collection container comprising:
a collection tube;
an interior reservoir formed within the collection tube, wherein the interior reservoir comprises:
an upper chamber,
a lower chamber, and
an elongated channel fluidly coupling the upper chamber and the lower chamber, wherein a diameter of the elongated channel is smaller than both a diameter of the upper chamber and a diameter of the lower chamber;
wherein an overall volume of the lower chamber is configured according to a total volume of a hematocrit portion of a maximum specimen sample volume collectable within the interior reservoir.

10. The specimen collection container of claim 9, wherein the elongated channel comprises an upper neck portion providing the transition between the upper chamber and the elongated channel.

11. The specimen collection container of claim 10, wherein the elongated channel further comprises a lower neck portion providing a transition between the lower chamber and the elongated channel.

12. The specimen collection container of claim 9, wherein the collection tube further comprises an open lip portion.

13. The specimen collection container of claim 12, wherein the open lip portion is at least partially formed as a scoop to accommodate the collection of a capillary blood sample into the interior reservoir.

14. A method of collecting and analyzing a capillary blood sample, the method comprising:
providing a specimen collection container comprising:
a collection tube;
an interior reservoir formed within the collection tube, wherein the interior reservoir comprises:
an upper chamber,
a lower chamber, and
an elongated channel fluidly coupling the upper chamber and the lower chamber, wherein a diameter of the elongated channel is smaller than both a diameter of the upper chamber and a diameter of the lower chamber;
collecting the capillary blood sample within the specimen collection container such that the lower chamber, the elongated channel, and at least a portion of the upper chamber are filled with the capillary blood sample;
subjecting the specimen collection container to centrifugation; and
inserting a bead-type separator into the specimen collection container after centrifugation in order to plug an upper neck portion of the elongated channel between the upper chamber and the elongated channel.

15. The method of claim 14, further comprising inserting a probe into the specimen collection container to collect a sample from a plasma/serum portion of the capillary blood sample.

16. The method of claim 15, wherein the probe is restricted from being inserted beyond the upper chamber of the specimen collection container by the bead-type separator.
